(19) United States
(12) Patent Application Publication
(10) Pub. No.: US 2024/0290483 A1
(43) Pub. Date: Aug. 29, 2024

(54) BIOMARKER PRIORITIZATION FOR EDGE COMPUTING RESOURCE MANAGEMENT

(71) Applicant: International Business Machines Corporation, Armonk, NY (US)

(72) Inventors: Paul R. Bastide, Ashland, MA (US); SAMIR NASSER, Durham, NC (US)

(21) Appl. No.: 18/174,731

(22) Filed: Feb. 27, 2023

(51) Int. Cl. G16H 40/63

(52) U.S. Cl. CPC G16H 40/63

(57) ABSTRACT

According to one embodiment, a method, computer system, and computer program product for biomarker prioritization is provided. The embodiment may include collecting data from a medical device associated with a user. The embodiment may also include identifying, for each type of data, at least one biomarker generated by the medical device associated with the user. The embodiment may further include associating each biomarker with a concerned component and/or a medical model. The embodiment may also include assigning a biomarker priority to a related component. The embodiment may further include generating a schedule for resource processing based on the biomarker priority. The embodiment may also include executing the schedule for resource processing.

### BACKGROUND

The present invention relates generally to the field of computing, and more particularly to edge computing.

Edge computing relates to a distributed information technology architecture where computing is performed on peripheral devices as opposed to centralized servers or cloud computing devices. In essence, edge computing moves storage and computing resources away from centralized facilities toward the data source itself. In this manner, edge computing may improve latency and efficiency more so than reliance on centralized servers, may carry security or privacy benefits, and may enable new networking paradigms that do not require internet-connected devices, or may improve efficiency of a local network.

With the addition of computing resources at the edge, data is collected, analyzed, and acted upon. Edge devices are often used in ultra-low latency and data sensitive applications. Often, edge computing pairs with sensors at an edge location to collect-analyze-act and feed forward the results to a centralized data center. In this sense, edge computing can be used in the context of the Internet of Things (IoT) to take advantage of the computing power of a variety of local computing devices, including not only computers, servers, and mobile phones, but also appliances, vehicles, and other IoT devices. These devices may also assist in network functionality, as may be the case in a mesh wireless network.

The healthcare field is quickly emerging as an industry with numerous uses for edge computing. Some initiatives are taking shape to build a patient-specific Internet of Medical Things (IoMT), which aggregates sensor data so real-time patient decisions may be made accurately and efficiently by medical professionals. Common areas such initiatives may be beneficial for include, but are not limited to, sports medicine, physical therapy and training, and personal healthcare.

IoMT allows caregivers and practitioners to monitor a remote patient's vital statistics using collected healthcare data, which is generally some form of biomarker, and act on the patient's care plan because of the collected and analyzed data. The data is generated, collected, analyzed, stored, retrieved, and managed using different mechanisms across different networks and applications allowing devices, such as mobile devices, ambulances, edge devices, and gateways, to all operate on the health care-related data.

However, in a computing environment with limited physical resources, a very clear need for the handing of certain concurrent requests exists. For example, in situations where the number of requests becomes greater than the processing capacity of the computing environment, a concern arises as to adverse, catastrophic results to patient health. In such situations, requests will likely require queuing which may lead to a degradation in quality of service (i.e., latency).

### SUMMARY

According to one embodiment, a method, computer system, and computer program product for biomarker prioritization is provided. The embodiment may include collecting data from a medical device associated with a user. The embodiment may also include identifying, for each type of data, at least one biomarker generated by the medical device associated with the user. The embodiment may further include associating each biomarker with a concerned component and/or a medical model. The embodiment may also include assigning a biomarker priority to a related component. The embodiment may further include generating a schedule for resource processing based on the biomarker priority. The embodiment may also include executing the schedule for resource processing.

In a preferred embodiment, the assignment is selected from a group consisting of expert-defined prioritization, inferred prioritization, and registered prioritization.

In a preferred embodiment, assigning a biomarker priority under inferred prioritization further includes, in response to multiple collections of a specific biomarker occurring during a given time window, averaging values of the specific biomarker.

In a preferred embodiment, the embodiment includes generating a future schedule based on predicted application usage and expected biomarker data usage and readings.

In a preferred embodiment, each biomarker priority is associated with a geofenced area or a GPS-derived activity.

In a preferred embodiment, the association is based on an inference of concern.

In a preferred embodiment, the at least one biomarker is identified using one or more of expert schema knowledge, profiling a schema, surveying a patient or schema, and device/application assertion.

### BRIEF DESCRIPTION OF THE SEVERAL VIEWS OF THE DRAWINGS

These and other objects, features and advantages of the present invention will become apparent from the following detailed description of illustrative embodiments thereof, which is to be read in connection with the accompanying drawings. The various features of the drawings are not to scale as the illustrations are for clarity in facilitating one skilled in the art in understanding the invention in conjunction with the detailed description. In the drawings:

FIG. 1 illustrates an exemplary networked computer environment according to at least one embodiment.

FIG. 2 illustrates an operational flowchart for a biomarker prioritization process according to at least one embodiment.

FIG. 3 illustrates a functional block diagram of biomarker prioritization computing layers according to at least one embodiment.

### DETAILED DESCRIPTION

Detailed embodiments of the claimed structures and methods are disclosed herein; however, it can be understood that the disclosed embodiments are merely illustrative of the claimed structures and methods that may be embodied in various forms. This invention may, however, be embodied in many different forms and should not be construed as limited to the exemplary embodiments set forth herein. In the description, details of well-known features and techniques may be omitted to avoid unnecessarily obscuring the presented embodiments.

It is to be understood that the singular forms “a,” “an,” and “the” include plural referents unless the context clearly dictates otherwise. Thus, for example, reference to “a component surface” includes reference to one or more of such surfaces unless the context clearly dictates otherwise.

Embodiments of the present invention relate to the field of computing, and more particularly to edge computing. The following described exemplary embodiments provide a system, method, and program product to, among other things, prioritize edge computing resources based on individual biomarkers. Therefore, the present embodiment has the capacity to improve the technical field of edge computing by improving data processing prioritization for edge computing, minimizing the likelihood of failed data process at the edge, and enhancing quality of service and responsiveness (i.e., latency).

As previously described, edge computing relates to a distributed information technology architecture where computing is performed on peripheral devices as opposed to centralized servers or cloud computing devices. In essence, edge computing moves storage and computing resources away from centralized facilities toward the data source itself. In this manner, edge computing may improve latency and efficiency more so than reliance on centralized servers, may carry security or privacy benefits, and may enable new networking paradigms that do not require internet-connected devices, or may improve efficiency of a local network.

With the addition of computing resources at the edge, data is collected, analyzed, and acted upon. Edge devices are often used in ultra-low latency and data sensitive applications. Often, edge computing pairs with sensors at an edge location to collect-analyze-act and feed forward the results to a centralized data center. In this sense, edge computing can be used in the context of the Internet of Things (IoT) to take advantage of the computing power of a variety of local computing devices, including not only computers, servers, and mobile phones, but also appliances, vehicles, and other IoT devices. These devices may also assist in network functionality, as may be the case in a mesh wireless network.

The healthcare field is quickly emerging as an industry with numerous uses for edge computing. Some initiatives are taking shape to build a patient-specific Internet of Medical Things (IoMT), which aggregates sensor data so real-time patient decisions may be made accurately and efficiently by medical professionals. Common areas such initiatives may be beneficial for include, but are not limited to, sports medicine, physical therapy and training, and personal healthcare.

IoMT allows caregivers and practitioners to monitor a remote patient's vital statistics using collected healthcare data, which is generally some form of biomarker, and act on the patient's care plan because of the collected and analyzed data. The data is generated, collected, analyzed, stored, retrieved, and managed using different mechanisms across different networks and applications allowing devices, such as mobile devices, ambulances, edge devices, and gateways, to all operate on the health care-related data.

However, in a computing environment with limited physical resources, a very clear need for the handing of certain concurrent requests exists. For example, in situations where the number of requests becomes greater than the processing capacity of the computing environment, a concern arises as to adverse, catastrophic results to patient health. In such situations, requests will likely require queuing which may lead to a degradation in quality of service (i.e., latency).

Biological markers, or biomarkers, relate to a category of medical signs indicative of a medical state or condition. Often measured through evaluation of blood, urine, and soft tissues, biomarkers have four common types: prognostic, predictive, pharmacodynamic, and surrogate. Through the various devices across the IoMT, patient biomarkers may be measured and monitored in real-time or at preconfigured periods for patients in need of such monitoring. However, computing devices or applications across the IoMT may demand differing amounts of resources depending on the biomarkers being calculated at any given time. As such, it may be advantageous to, among other things, prioritize computing power across an edge computing environment to prioritize resources necessary to measure the various biomarkers indicative of various physical, biological, or chemical conditions for a patient.

According to one embodiment, a biometric prioritization program may collect medical data device data to identify biomarkers for a patient. The biomarkers may be associated with an associated component and/or model of particular concern and assigned a priority related to the assigned component or model. The biometric prioritization program may then generate and execute a schedule for resource processing based on the assigned biomarker prioritization.

Any advantages listed herein are only examples and are not intended to be limiting to the illustrative embodiments. Additional or different advantages may be realized by specific illustrative embodiments. Furthermore, a particular illustrative embodiment may have some, all, or none of the advantages listed above.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

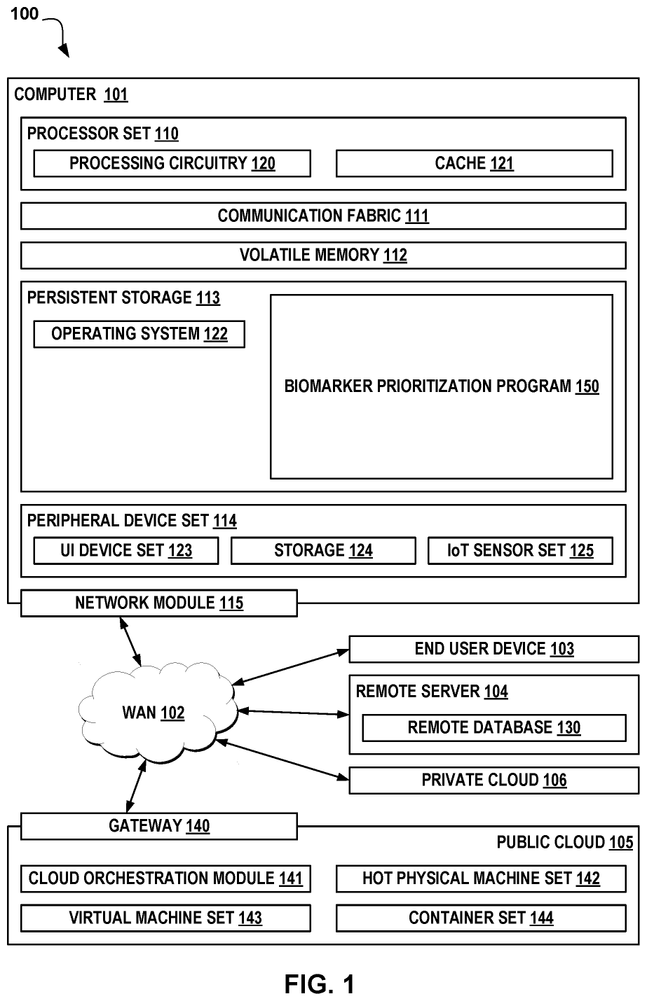

Referring now to FIG. 1, computing environment 100 contains an example of an environment for the execution of at least some of the computer code involved in performing the inventive methods, such as biomarker prioritization program 150. In addition to biomarker prioritization program 150, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and biomarker prioritization program 150, as identified above), peripheral device set 114 (including user interface (UI), device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

Computer 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, for illustrative brevity. Computer 101 may be located in a cloud, even though it is not shown in a cloud in FIG. 1. On the other hand, computer 101 is not required to be in a cloud except to any extent as may be affirmatively indicated.

Processor set 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in biomarker prioritization program 150 in persistent storage 113.

Communication fabric 111 is the signal conduction path that allows the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

Volatile memory 112 is any type of volatile memory now known or to be developed in the future. Examples include dynamic type random access memory (RAM) or static type RAM. Typically, the volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

Persistent storage 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid-state storage devices. Operating system 122 may take several forms, such as various known proprietary operating systems or open-source Portable Operating System Interface-type operating systems that employ a kernel. The code included in biomarker prioritization program 150 typically includes at least some of the computer code involved in performing the inventive methods.

Peripheral device set 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made though local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

Network module 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN 102 and/or LANs typically include computer hardware such as copper transmission cables, optical transmission fibers, wireless transmission, routers, firewalls, switches, gateway computers and edge servers.

End user device (EUD) 103 is any computer system that is used and controlled by an end user and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

Remote server 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

Public cloud 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

Private cloud 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community, or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

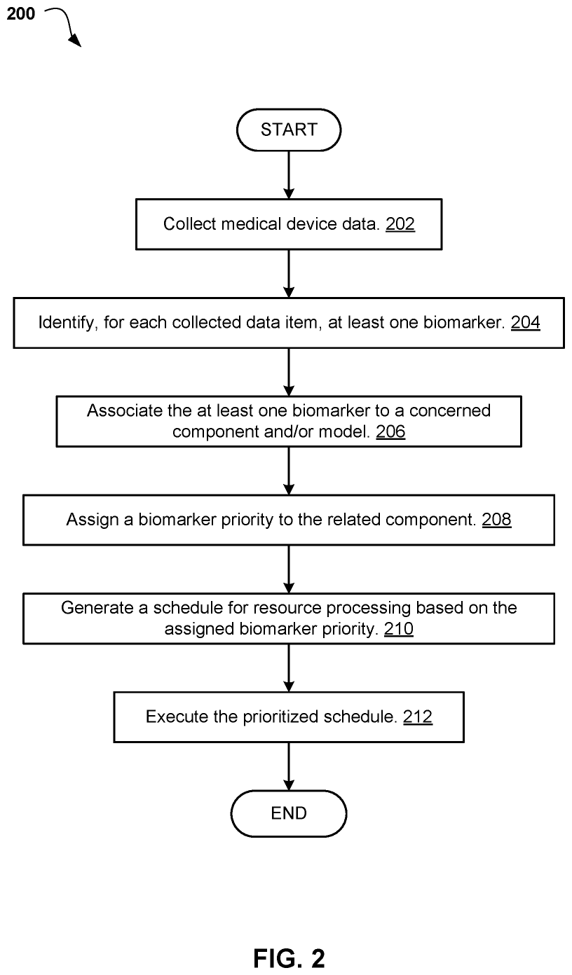

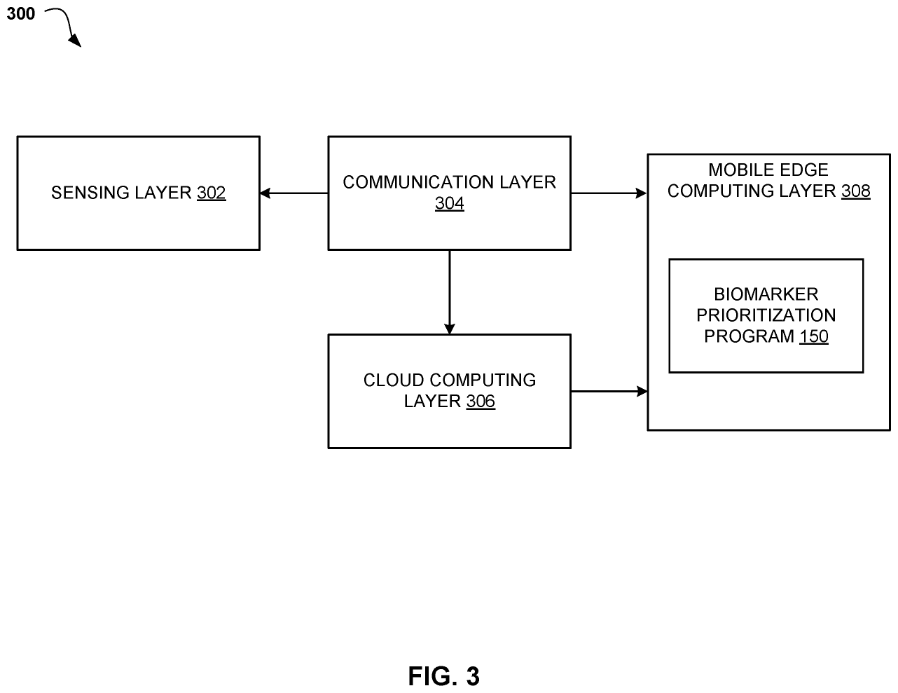

According to at least one embodiment, the biomarker prioritization program 150 may utilize captured medical device information to identify at least one biomarker relevant to each item of medical device information. The biomarker prioritization program 150 may then associated each identified biomarker with a model or component related to tracking the biomarker. The biomarker prioritization program 150 may assign a priority to each component based on a period of reporting the biomarker to a user. The biomarker prioritization program 150 may then generate and execute a schedule for resource processing based on the assigned biomarker priority. Furthermore, notwithstanding depiction in computer 101, the biomarker prioritization program 150 may be stored in and/or executed by, individually or in any combination, end user device 103, remote server 104, public cloud 105, and private cloud 106. The biomarker prioritization method is explained in more detail below with respect to FIGS. 2 and 3.

Referring now to FIG. 2, an operational flowchart for a biomarker prioritization process 200 is depicted, according to at least one embodiment. At 202, the biomarker prioritization program 150 collects medical device data. Typically, medical devices for capture-analyze-act processing utilize one of three common profiles: localized devices, tethered devices, or data feeder and optimization devices. Localized medical devices perform data capture and analysis within the device itself and are fit for the single or limited set of alerts to act on, such as an inhaler which reports the number of doses given. Tethered medical devices include a localized profile but expanded upon that profile through a paired connection, such as a wireless pairing of an inhaler to a smartphone. Wireless connections utilized by tethered medical devices to pair with other devices include, but are not limited to, Wi-Fi, RFID, radio transmission, near-field communication (NFC), and Bluetooth® (Bluetooth and all Bluetooth-based trademarks and logos are trademarks or registered trademarks of Bluetooth SIG, Inc. and/or its affiliates). Data feeder and optimization devices transmit captured information directly to a central repository for storage and processing. Common examples may include pacemakers.

The biomarker prioritization program 150 may mainly utilize localized medical devices and data feeder and optimization devices. However, in one or more other embodiments, the biomarker prioritization program 150 may also utilize tethered medical devices either exclusively or in any combination with localized devices and data feeder and optimization devices. Regardless, the biomarker prioritization program 150 may utilize any medical device capable of characterization as computer 101 or a device within IoT sensor set 125.

The biomarker prioritization program 150, installed on or instructing the medical device, may collect real-time data and execute rudimentary processing on the medical device itself. The biomarker prioritization program 150 may instruct the medical device to synchronize with any tethered device, such as a smartphone, in near real-time since a real-time feed may seldom be available. However, in embodiments where a real-time feed is available, the biomarker prioritization program 150 may perform synchronization in real time. The biomarker prioritization program 150, through the medical device, may synchronize or forward collected data on preconfigured intervals, or blocks. The synchronization blocks may contain the most recent data captures with assigned timelines in an ordered array, such as:

```
[
  ...
  {
    “type”: “pulse-ox”,
    “reading”: 90.0
    “date”: “2022-12-1T08:31:10”,
    “nonce”: “12-13-4-5”
  }
  ...
]
```

The biomarker prioritization program 150 may rely on unique identification of the data elements using a nonce or generated UUID for each data element in the synchronized block. The nonce ensures unique readings are not read or treated improperly.

In one or more embodiments, the biomarker prioritization program 150 may act as a shim between the medical device, the data receiving application, and any analytics performed by an analytical model. The biomarker prioritization program 150 may store each received data element in a global temporary table that may be backed up by local persistence. As an example, the global temporary table for the previous scenario may appear as described below in Table 1.

TABLE 1

| Name | Value | Description |
|---|---|---|
| Application | pulse-ox | The Application of device |
| | | name that generated the |
| | | reading. |
| Date | “2022-12-1T08:31:10” | Date and Time of Data |
| | | Generation |
| Unique ID | “12-13-4-5” | The device asserted unique |
| | | id, which may be prepended |
| | | on the unique-id to help with |
| | | selectivity. |
| Biomarker_link_id | n/a | A generated id to link to the |
| | | biomarkers via an association |
| Data | <<blob of above json>> | The raw data (ideally in an |
| | | open format such as protobuf |
| | | or JSON). However, the |
| | | Shim may include an |
| | | additional layer that does an |
| | | intermediate translation so |
| | | the data can be anlayzed. |

Next, at 204, the biomarker prioritization program 150 identifies at least one biomarker for each collected data item. The biomarker prioritization program 150 may identify the biomarkers in one or more ways, such as, but not limited to, expert knowledge, profiling the schema, surveying a patient or schema, and device/application assertion. Device/application assertions may include, but are not limited to, metadata on the process that is currently running (e.g., labels on the running process that help identify resource usage), assertions on a package of the application (e.g., similar to privacy labels), explicit subscriptions to device data (e.g., allowing access to user data, such as telematics, steps, and heartrate, from a specific step-tracker application), and correlated events (e.g., heart rate readying from a device and a fitness application that shows a correlation between user heart rate when the fitness application is open). In at least one embodiment, the number of biomarkers identified by the biomarker prioritization program 150 may be preconfigured to a limited number rather than an ongoing, infinite number. Such limitation may reduce the amount of analysis done from the incoming, captured data. Furthermore, if the biomarker prioritization program 150 receives data outside of a preconfigured time window, the biomarker prioritization program 150 may remove or omit such data from the collected pool. For example, a synchronized cardiological reading that is out of date due to a reboot of a smartphone may be discarded or marked as ignored.

The biomarker prioritization program 150 may identify at least one biomarker for each collected data item by first identifying expert schema knowledge. The biomarker prioritization program 150 may identify the expert schema knowledge by analyzing data elements for a schema definition for a specific application. For example, continuing the previous situation with respect to Table 1, the biomarker prioritization program 150 may identify the schema field for “reading” as being associated with the “OXYGEN” biomarker. The biomarker prioritization program 150 may ideally utilize this analysis for open standards, such as HL7v3, HL7v2, and FHIR v4/v5. In one or more embodiments, the biomarker prioritization program 150 may combine biomarkers in chains such as, when biomarkers are related (e.g., vascular biomarkers and pulse-ox biomarkers).

Once the schema is identified, the biomarker prioritization program 150 may profile the schema. The natural language names of fields may be profiled for key words. For example, “reading.temp” may be utilized as a candidate for association with the value “Temperature” since the term “reading” may be too general for a proper candidate. The biomarker prioritization program 150 may profile further on subsequent value types.

Surveying may relate to prompting a user to provide information related to various biomarkers. The biomarker prioritization program 150 may survey the patient or practitioner to identify biomarkers. While surveying may be lossy, the biomarker prioritization program 150 may utilize the surveyed information to confirm or survey an existing hypothesis on the biomarkers.

In one or more embodiments, the biomarker prioritization program 150 may also enable applications to assert the biomarkers used in a two-way, ask-answer manner that results in a biomarker identification.

In one or more other embodiments, the biomarker prioritization program 150 may profile natural language of application documents. The profiling introspection may extend into models running on edge computing devices such that a biomarker is inferred based on the model's name or temporal running of the model.

Then, at 206, the biomarker prioritization program 150 associates the at least one biomarker to a concerned component and/or model. The biomarker prioritization program 150 may associate biomarkers to a concerned component and/or model using an inference of concern or a registered concern. An inference of concern may relate to an activity after the data being retrieved is captured. For example, an individual's pulse-ox readings may synchronize with a device and, then, after a period of time a local pulmonary health model is executed. The model may generate an association, such as Pulse-Ox-Data {reading.temp} 15-min Pulmonary Health Model. The more data gathered may result in more associations and may allow for a stronger inference to be made. A registered concern may relate to marking an association between data items when particular terms are present. For example, the biomarker prioritization program 150 may associate any biomarker with “temp” and “oxygen” to a model titled “Pulmonary Health Model”. For either an inference of concern or a registered concern, the biomarker prioritization program 150 may associate the biomarkers with the components using an association table. For example, the associations may be depicted as seen in Table 2.

TABLE 2

| Biomarker | Connects-To |
|---|---|
| Oxygen | Pulmonary Model |
| Temp | Pulmonary Model |

The associations, as depicted in Table 2, may help prioritize and contain a third column including an asserted priority for each biomarker. Furthermore, the biomarker priorities may be associated with a geofenced area or a GPS-derived activity, such as walking, going up the stairs, and leaving a residence. The biomarker prioritization program 150 may correlate such location data from a location-capable device (e.g., a smartphone) or through triangulation of a the current location using wireless access points. The biomarker prioritization program 150 may associate the locations with a map in a latitude-longitude-altitude coordinate system to help pinpoint an exact location within an area. Once the locations are pinpointed, the biomarker prioritization program 150 may correlate the locations with an immediate history of action to determine speed and relative activity. For example, if a user moves from a 1,1,1 coordinate to a 1,2,10 coordinate, the biomarker prioritization program 150 may infer that the user is traversing a set of stairs as altitude is gaining significantly. Similarly, if a user moves from 1,10,1 to 1,100,1, the biomarker prioritization program 150 may infer the user is engaging in vigorous activity since the user traversed 90 steps in less than 60 seconds.

Next, at 208, the biomarker prioritization program 150 assigns a biomarker priority to the related component. The biomarker prioritization program 150 may assign a biomarker priority in one or more manners, such as, but not limited to, a expert-defined, inferred, and registered. Expert-defined prioritization may relate to prioritizing biomarkers based on expert determination. For example, a doctor may decide the cardiovascular process biomarker (e.g., “telemetry”) may be more important than a pulmonary process biomarker (e.g., “oxygen”) or a temperature-related biomarker (e.g., “temperature”). Inferred prioritization may relate to prioritizing biomarkers based on a number of times the model or application ran or executed during a given window of time. The biomarker prioritization program 150 may use a rolling window, a fixed window, or never reset the window. If multiple collections of the same biomarker are used or received during a given window, the biomarker prioritization program 150 may average values for that biomarker rather than accumulate the biomarkers. Registered biomarkers relate to biomarkers that are prioritized by a specific application or model such that the model states how important to life-saving the biomarker is. If the biomarker is captured, a downstream model may then take precedence over less important biomarker application associations. In one or more embodiments, the biomarker prioritization program 150 may perform prioritization based on the assignments without needing external intervention, however, an external override may be available. In one or more other embodiments, the biomarker prioritization program 150 may share assigned biomarker priorities with a priority or scheduling component, which may aggregate and sort the priority scheduling. Furthermore, when assigning the prioritizations, the biomarker prioritization program 150 may prioritize resources to specific analytics models, infrastructure, or CPU limits.

The biomarker prioritization program 150 may also add a transient priority factor based on one or more of factors including, but not limited to, a patient's condition, severity of condition, and correlation to the biomarker; acute need (e.g., whether the patient is in an urgent-care or at-home with a chief complaint), and patient or caregiver assertion that a condition, and thus biomarkers, have changed. Further examples related to acute need may include a doctor sending a request to retrieve patient data needed to decide about a patient who has a life-threatening condition or a doctor sending a request to get the mobile edge computing layer to perform some time-consuming analytics about a patient who does not have serious conditions.

In at least one embodiment, the biomarker prioritization program 150 may store the assignments in a lookup table either globally or associated with a biomarker or with a specific model, such as depicted in Table 3.

TABLE 3

| Application | Priority | Type | Biomarker |
|---|---|---|---|
| Pulmonary Model | 1 | Inferred | Temperature |
| Pulmonary Model | 1000 | Registered | Oxygen |

In another embodiment, the biomarker prioritization program 150 may consider a geofence to switch prioritized schedules. The geofence may be a proxy for the activity which is associated with an important biomarker. For example, when a patient is walking up a flight of stairs, lung function-related biomarkers, such as pulse-ox levels, may be most important.

In yet another embodiment, when determining the prioritization of biomarkers, the biomarker prioritization program 150 may consider the workload placement based on the biomarkers present in the data. For example, a patient's heart rate measurement may be considered very important to his doctor and, therefore, the workload may process at the edge location versus the glucose monitoring, which may be processed later at the doctor's location. The biomarker prioritization program 150 may then use the application with all historical data versus only a partial data set with incomplete knowledge.

Then, at 210, the biomarker prioritization program 150 generates a schedule for resource processing based on the assigned biomarker priority. The biomarker prioritization program 150 may generate code or a model of a schedule based on the assigned priority. For example, the schedule may be generated as:

SELECT SUM(PRIORITY) PRIORITY, APPLICATION
FROM BIOMARKER_PRIORITY
JOIN ASSOCIATES_TO on ASSOCIATE_TO.CONNECTS_to =
BIOMARKER_PRIORITY.Biomarker
JOIN DATA_TABLE on DATA_TABLE.BIOMARKER_LINK_ID =
ASSOCIATES_TO.BIOMARKER
GROUP BY BIOMARKER_PRIORITY.PRIORITY
SORT BY PRIORITY

In one or more embodiments, the biomarker prioritization program 150 may limit the number of models running related to a specific biomarker to be a unique group. The biomarker prioritization program 150 may consider a subsequent rescheduling of a second set of model to run exclusive to the first run. For example, the biomarker prioritization may consider an application with a heartrate biomarker, an application with a pulse-ox biomarker, and a model with a heartrate biomarker. The application and model with the heartrate biomarker may be prioritized as a group of components versus the group with the pulse-ox biomarker.

In one or more other embodiments, the biomarker prioritization program 150 may generate a future schedule based on predicted application usage and expected biomarker data usage and readings. For example, if a user calendar indicates an outdoor activity later in the day and the medical device monitoring is programmed to capture measurements when a user is active, the biomarker prioritization program 150 may generate a future schedule to capture appropriate biomarkers during the predicted, future activity.

Next, at 212, the biomarker prioritization program 150 executes the prioritized schedule. As soon as an application or model requests computing resources, the biomarker prioritization program 150 may execute the schedule to run the set of scheduled processes and filter the requested computer resources to ensure the appropriate resources as allocated for the appropriate processes. In one or more embodiments, the biomarker prioritization program 150 may filter or limit the requested computer resources based on the generated schedule. The biomarker prioritization program 150 may allow only a small set of the overall generated schedule (e.g., limit five processes at a time) to avoid over burdening overall system performance. For example, the schedule may generate a list of one hundred open component models and applications and generate a priority list, rather than serially process the one hundred components, the biomarker prioritization program 150 may allow a subset of five to simultaneously execute, then generate a new schedule. In alternate embodiments, the biomarker prioritization program 150 may select five, finish the five, and then the next five until the complete list of requests for resources are fulfilled.

Referring now to FIG. 3, an exemplary block diagram of the biomarker prioritization computing layers 300 is depicted according to at least one embodiment. In one or more embodiments, an overall system architecture may include a sensing layer 302, communication layer 304, cloud computing layer 306, and mobile edge computer layer 308. The mobile edge computing layer may host the biomarker prioritization program 150. In one or more embodiments, sensing layer 302, communication layer 304, cloud computing layer 306, and mobile edge computer layer 308 may be on the same device, such as computer 101, or spanned across tethered devices.

The sensing layer 302 may include sensing-type devices, such as pressure sensors, oxygen sensors, or any sensor included in IoT sensor set 125, capable of data capture. Typically, devices within sensor layer 302 may be located in various locations amenable to healthcare monitoring, such as, but not limited to, a home healthcare setting, hospitals, and ambulances. The communication layer 304 may include devices mainly intended for communication, including, but not limited to, cell phones, personal digital assistant devices or any device capable of communicating across a network, and capable of non-intensive data processing and analytics. Typically, devices within communication layer 304 may be located in various locations where the processing of data may be performed at the edge, such as, but not limited to, a doctor's office, hospitals, and a patient's residence. The cloud computing layer 306 may be capable of scalable computing power, intensive data processing, analytics, and data storage. More specifically, the cloud computing layer 306 may perform capabilities inefficient or unfeasible of performance by sensing layer 302 or communication layer 304.

Mobile edge computing layer 308, which may host the biomarker prioritization program 150, may possess cross functionality exhibited by communication layer 304 and cloud computing layer 308. More specifically, mobile edge computing layer 308 may be capable of constrained computing power, substantial data process, calculate analytics. Furthermore, the mobile edge computing layer 308 may be capable of data storage and be latency sensitive.

It may be appreciated that FIGS. 2 and 3 provide only an illustration of one implementation and do not imply any limitations with regard to how different embodiments may be implemented. Many modifications to the depicted environments may be made based on design and implementation requirements. For example, a patient under the care of a doctor due to a diagnosis of diabetes and asthma may utilize a Bluetooth®-enabled pulse-oximeter and continuous glucose monitoring (CGM) to record statistics related to both conditions. The patient's smartphone (e.g., end user device 103) may be paired with the corresponding medical devices. The patient's smartphone may also have several care management applications installed such as APP-1, a diabetes management application concerned with CGM readings; APP-2, a diabetes management application concerned with CGM and temperature readings; APP-3, a pulmonary application concerned with oxygen levels and heart rate readings; and APP-4, an application that forwards data feeds to the patient's primary caregiver. Periodically, the CGM may report blood sugar level to the patient's smartphone through the Bluetooth® pairing. The patient may also check pulse-ox levels after exercise and synchronize the data to the paired smartphone.

In one situation, the patient may climb a set of stairs and the paired pulse-oximeter may record a pulse-ox level of 90%. The biomarker prioritization program 150 may capture the medical device data (DATA-1) from the pulse-oximeter as:

```
{
  “type”: “pulse-ox”,
  “oxygen”: 90.0,
  “rate”: 130.0,
  “temp_c”: 25.0,
  “date”: “2022-12-1T08:31:10”,
  “uuid”: “12-13-4-5”
}
```

The biomarker prioritization program 150 may then identify DATA-1 has “OXYGEN”, “HEART RATE”, and “BODY_TEMPERATURE” as biomarkers. The biomarker prioritization program 150 may then associate the identified biomarkers to a concerned component and/or model. For example, the biomarker prioritization program 150 may associate CGM readings, which may be valid for five second, with App-1 and App-2, temperature readings, which may be valid for one hour, with App-2, oxygen level readings, which may be valid for 15 minutes, with App-3 and App-4, and heart rate readings, which may be valid for one minute, with App-3 and App-4. In one or more other embodiments, the biomarker prioritization program 150 may allow for an administrator, such as a clinician, to perform additional prioritization of the biomarkers based on administrator determined priorities.

Using the associations, the biomarker prioritization program 150 may assign each biomarker priority to the associated component (i.e., application or model). For example, App-1 may have CGM readings of the previous five seconds assigned, App-2 may have temperature readings from the previous hour and CGM readings from the previous five seconds assigned, App-3 may have oxygen level readings from the previous 15 minutes and hear rate readings from the previous one minutes assigned, and App-4 may have oxygen level readings from the previous 15 minutes and hear rate readings from the previous one minutes assigned.

The biomarker prioritization program 150 may then generate a schedule for resource processing based on the assigned biomarker priority. For example, the biomarker prioritization program 150 establish a schedule for resource processing that prioritizes applications as App-2, App-1, App-3, and App-4 due to the resources needed for processing by each application before the respective biomarker data expires. Therefore, the biomarker prioritization program 150 may execute the prioritized schedule and conserves resources for the most important applications based on the biomarker importance. For example, App-2 executes its analysis and App-1, App-3, and App-4 wait in queue to process, which may be a beneficial use of resources in a resource-constrained environment.

In other embodiments, the biomarker prioritization program 150 may federate multiple users' data (e.g., patient data) together to determines which biomarkers take priority. Such federation may be appropriate when calculating and tracking group biomarkers rather than biomarkers for a single individual as described in multiple situations above.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A processor-implemented method, the method comprising:
collecting data from a medical device associated with a user;
identifying, for each type of data, at least one biomarker generated by the medical device associated with the user;
associating each biomarker with a concerned component and/or a medical model;
assigning a biomarker priority to a related component;
generating a schedule for resource processing based on the biomarker priority; and
executing the schedule for resource processing.

2. The method of claim 1, wherein the assignment is selected from a group consisting of expert-defined prioritization, inferred prioritization, and registered prioritization.

3. The method of claim 2, wherein assigning a biomarker priority under inferred prioritization further comprises:
in response to multiple collections of a specific biomarker occurring during a given time window, averaging values of the specific biomarker.

4. The method of claim 1, further comprising:
generating a future schedule based on predicted application usage and expected biomarker data usage and readings.

5. The method of claim 1, wherein each biomarker priority is associated with a geofenced area or a GPS-derived activity.

6. The method of claim 1, wherein the association is based on an inference of concern.

7. The method of claim 1, wherein the at least one biomarker is identified using one or more of expert schema knowledge, profiling a schema, surveying a patient or schema, and device/application assertion.

8. A computer system, the computer system comprising:
one or more processors, one or more computer-readable memories, one or more computer-readable tangible storage medium, and program instructions stored on at least one of the one or more tangible storage medium for execution by at least one of the one or more processors via at least one of the one or more memories, wherein the computer system is capable of performing a method comprising:
collecting data from a medical device associated with a user;
identifying, for each type of data, at least one biomarker generated by the medical device associated with the user;
associating each biomarker with a concerned component and/or a medical model;
assigning a biomarker priority to a related component;
generating a schedule for resource processing based on the biomarker priority; and
executing the schedule for resource processing.

9. The computer system of claim 8, wherein the assignment is selected from a group consisting of expert-defined prioritization, inferred prioritization, and registered prioritization.

10. The computer system of claim 9, wherein assigning a biomarker priority under inferred prioritization further comprises:
in response to multiple collections of a specific biomarker occurring during a given time window, averaging values of the specific biomarker.

11. The computer system of claim 8, wherein the method further comprises:
generating a future schedule based on predicted application usage and expected biomarker data usage and readings.

12. The computer system of claim 8, wherein each biomarker priority is associated with a geofenced area or a GPS-derived activity.

13. The computer system of claim 8, wherein the association is based on an inference of concern.

14. The computer system of claim 8, wherein the at least one biomarker is identified using one or more of expert schema knowledge, profiling a schema, surveying a patient or schema, and device/application assertion.

15. A computer program product, the computer program product comprising:
one or more computer-readable tangible storage medium and program instructions stored on at least one of the one or more tangible storage medium, the program instructions executable by a processor capable of performing a method, the method comprising:
collecting data from a medical device associated with a user;
identifying, for each type of data, at least one biomarker generated by the medical device associated with the user;
associating each biomarker with a concerned component and/or a medical model;
assigning a biomarker priority to a related component;
generating a schedule for resource processing based on the biomarker priority; and
executing the schedule for resource processing.

16. The computer program product of claim 15, wherein the assignment is selected from a group consisting of expert-defined prioritization, inferred prioritization, and registered prioritization.

17. The computer program product of claim 16, wherein assigning a biomarker priority under inferred prioritization further comprises:
in response to multiple collections of a specific biomarker occurring during a given time window, averaging values of the specific biomarker.

18. The computer program product of claim 15, wherein the method further comprises:
generating a future schedule based on predicted application usage and expected biomarker data usage and readings.

19. The computer program product of claim 15, wherein each biomarker priority is associated with a geofenced area or a GPS-derived activity.

20. The computer program product of claim 15, wherein the association is based on an inference of concern.
